SEQUENCE LISTING (2 sequences)

SEQ ID NO: 1 - protein, 543 aa, Homo sapiens
MSRESDVEAQQSHGSSACSQPHGSVTQSQGSSSQSQGISSSSTSTMPNSSQSSHSSSGTLSSLETVSTQELYSIPEDQEPEDQEPEEPTPAPWARLWALQDGFANLECVNDNYWFGRDKSCEYCFDEPLLKRTDKYRTYSKKHFRIFREVGPKNSYIAYIEDHSGNGTFVNTELVGKGKRRPLNNNSEIALSLSRNKVFVFFDLTVDDQSVYPKALRDEYIMSKTLGSGACGEVKLAFERKTCKKVAIKIISKRKFAIGSAREADPALNVETEIEILKKLNHPCIIKIKNFFDAEDYYIVLELMEGGELFDKVVGNKRLKEATCKLYFYQMLLAVQYLHENGIIHRDLKPENVLLSSQEEDCLIKITDFGHSKILGETSLMRTLCGTPTYLAPEVLVSVGTAGYNRAVDCWSLGVILFICLSGYPPFSEHRTQVSLKDQITSGKYNFIPEVWAEVSEKALDLVKKLLVVDPKARFTTEEALRHPWLQDEDMKRKFQDLLSEENESTALPQVLAQPSTSRKRPREGEAEGAETTKRPAVCAAVL

SEQ ID NO: 2 - protein, 142 aa, Homo sapiens
SGRGKTGGKARAKAKSRSSRAGLQFPVGRVHRLLRKGHYAERVGAGAPVYLAAVLEYLTAEILELAGNAARDNKKTRIIPRHLQLAIRNDEELNKLLGGVTIAQGGVLPNIQAVLLPKKTSATVGPKAPSGGKKATQASQEY

(19) United States
(12) Patent Application Publication
(10) Pub. No.: US 2008/0253632 A1
(43) Pub. Date: Oct. 16, 2008

(54) Method of Detecting Precancerous Lesions

(76) Inventor: Thanos D. Halazonetis, Geneva (CH)

Correspondence Address: PEPPER HAMILTON LLP, ONE MELLON CENTER, 50TH FLOOR, 500 GRANT STREET, PITTSBURGH, PA, 15219, US

(21) Appl. No.: 11/910,016

(22) PCT Filed: Mar. 30, 2006
(86) PCT No.: PCT/US06/11377

§ 371 Date: Sep. 28, 2007

Related U.S. Application Data
(60) Provisional application No. 60/666,402, filed on Mar. 30, 2005.

(51) Int. Cl. G06K 9/00

(52) U.S. Cl. 382/129

(57) ABSTRACT

The present invention relates to methods of detection of precancerous lesions and/or cancer. The present invention also relates to the presence of DNA replication stress in precancerous lesions. The present invention further relates to the detection of loss of heterozygosity at common fragile sites and phosphorylated substrates of DNA damage activated kinases.

### CROSS-REFERENCE TO RELATED APPLICATIONS

The present application claims priority to U.S. Provisional Application Ser. No. 60/666,402, filed Mar. 30, 2005, which is hereby incorporated by reference in its entirety.

### GOVERNMENT SUPPORT

This invention was made with U.S. Government support (NIH Grant No. 2 R01 CA 76367) and the U.S. Government may therefore have certain rights in the invention.

### BACKGROUND

Cancer that is detected early often leads to high remission rates and longer survival times in patients. Although, many tests have been developed to identify and/or detect cancer at its earliest stage, there is still a long-felt need for new and improved markers of cancer to detect cancer and precancerous lesions even earlier.

Many methods exist to diagnose cancer. If a patient is suspected of having cancer, then a biopsy may be performed and tissue sections may be examined under the microscope, This histology analysis is often sufficient to lead to a diagnosis of cancer, because cancer tissue in most cases can be readily distinguished from normal tissue under the microscope. However, if the biopsied tissue only harbors a precancerous lesion, then the diagnosis is more difficult. Precancerous lesions may be hard to distinguish from normal tissue under the microscope, Further, certain non-precancerous lesions, such as hyperplasias and dysplasias induced by chronic inflammation or hormonal stimuli may look exactly like precancerous lesions under the microscope. Thus, there is a need for methods to aid in the diagnosis of precancerous lesions and this invention is indeed focused on precancerous lesions. The present invention fulfills this need as well as others.

### SUMMARY OF THE INVENTION

In some embodiments, the present invention provides methods of determining the presence of precancerous hyperplastic and/or dysplastic lesions and/or distinguishing precancerous hyperplastic and/or dysplastic lesions from non-precancerous tissue comprising determining the phosphorylation status of a substrate of ATM and/or ATR, wherein the presence of a phosphorylated ATM and/or ATR substrate indicates the presence of a precancerous lesion.

In some embodiments, the present invention provides methods determining the presence of precancerous hyperplastic and/or dysplastic lesions and/or distinguishing precancerous hyperplastic and/or dysplastic lesions from non-precancerous tissue comprising determining the presence of DNA replication stress in the tissue, wherein the presence of DNA replication stress indicates the presence of a precancerous lesion.

In some embodiments, the present invention provides methods determining the presence of precancerous hyperplastic and/or dysplastic lesions and/or distinguishing precancerous hyperplastic and/or dysplastic lesions from non-precancerous tissue comprising detecting in a sample loss of heterozygosity (LOH) at a common fragile site wherein a LOH at the common fragile site indicates the presence of a precancerous lesion.

In some embodiments, the present invention provides kits for the detection of a precancerous lesion comprising at least one antibody to detect the phosphorylation status of a substrate of ATM and/or ATR or primers to detect LOH of a common fragile site.

### BRIEF DESCRIPTION OF DRAWINGS

FIG. 1. Activation of the DNA double strand break (DSB) checkpoint pathway in human preneoplastic and neoplastic lesions. A. Current model of DNA DSB signaling pathway leading to p53-dependent apoptosis. For clarity only a subset of the DNA DSB response proteins are shown. B. Immunohistochemistry of normal bronchial epithelium (Normal), hyperplastic (Hyperpl.), dysplastic (Dyspl.) and non-small cell lung carcinoma (NSCLC) lesions. Ki67 and TUNEL staining were used to calculate proliferation and apoptotic indices, respectively. γ-H2AX, phosphorylated H2AX; pT68, Chk2 phosphorylated on Thr68. The distribution of 53BP1 in cells was examined by immunofluorescence (IF): 53BP1, green; blue, outline of the nuclei. p53 wt and p53mut, wild-type and mutant p53 genotypes, respectively. C. Immunohistochemistry of a NSCLC that does not express 53BP1. Upper panel: section showing normal epithelium (ep), stroma (str) and NSCLC (ca) stained for 53BP1. Lower panels: adjacent NSCLC sections stained for γ-H2AX and Chk2 phosphorylated on Thr68 (pT68). D. Reverse transcriptase-polymerase chain reaction (RT-PCR) and immunoblot (IB) analysis of two NSCLC lesions that stained negative for 53BP1 by immunohistochemistry. gapdh, glyceraldehyde-3-phosphate dehydrogenase; N, normal tissue; T, tumor tissue. E. Immunohistochemistry of a dysplastic nevus (Dyspl.) and melanoma (Melan.) from the same patient. F. Immunohistochemistry of two areas of normal skin adjacent to the dysplastic nevus shown in (e). One of the areas of normal skin (lower panels) corresponds to a freckle (simple lentigo). S100 staining marks the melanocytes. G. Immunohistochemistry of normal colonic epithelium and colon carcinoma (CA) from the same patient. H. Summary of DNA DSB responses in human normal tissues, preneoplastic and neoplastic lesions. The numbers in parentheses indicate the number of samples examined. The bars show the mean and standard deviation of the percentage of cells that stained positive for the indicated marker.

FIG. 2. Activation of the DNA DSB checkpoint pathway in experimentally-induced hyperplasias. Human newborn foreskin or adult skin xenografts were either untreated (control, Ctl) or injected with an adenoviral vector that expresses green fluorescent protein (GFP) or with adenoviral vectors overexpressing growth factors (GF). A. Cross-section of the entire epithelium of control and growth factor-treated adult skin xenografts from the same donor (#5) stained for γ-H2AX. B. Immunohistochemistry of control and growth factor-treated adult skin xenografts from donor #5 for Ki67, Chk2 phosphorylated on Thr68 (pT68) and p53. TUNEL staining was used to mark the apoptotic cells and immunofluorescence (IF) was used to monitor the intracellular localization of 53BP1. C. Immunoblot analysis of extracts prepared from control and growth factor-treated adult skin xenografts from donors #3 and #4 to monitor H2AX and Chk2 phosphorylation (γ-H2AX and pT68, respectively) and p53 protein levels. D. Telomere length of control and growth factor-treated adult skin xenografts from donors #3 and #5 monitored by Southern blot analysis. The erythroleukemia cell line K562 served as a control. E. Summary of DNA DSB responses in newborn foreskin and adult skin xenografts. The numbers in parentheses indicate the number of xenografts examined. The bars show the mean and standard deviation of the percentage of cells in each xenograft that stained positive for the indicated marker.

FIG. 3. Allelic imbalance at common fragile sites in early human cancer lesions and model for activation of the DNA DSB checkpoint in cancer. A. Allelic imbalance at the common fragile site FRA3B on chromosome 3 p14 is frequent in lung hyperplasias and precedes allelic imbalance at other loci. Allelic imbalance analysis of normal bronchial epithelium, hyperplastic and neoplastic tissue from 11 NSCLC patients. The microsatellite markers used in the analysis and their chromosomal position are indicated on the left. Markers D3S1289 and D3S1300 map to FRA3B; marker D3S1263 maps to 3p25; and marker D3S1566 maps to 3 p13. Allelic Imbal., allelic imbalance; Micros. Inst., microsatellite instability; Non-inform., non-informative loci (homozygous). B. Allelic imbalance at the common fragile site FRA3B in human adult skin xenografts induced to become hyperplastic by adenoviruses expressing growth factors. Means and standard errors of allele ratios for microsatellite markers D3S1263, D3S1289, D3S1300 and D3S1566 from control (Ctl) and growth factor-treated (GF) xenografts from donors #3, #4 and #5. Ratios are not shown for non-informative (homozygous) loci. The allele ratios were determined from 2 independent polymerase chain reactions per sample and scaled so the ratio would be 1 for the control xenografts. Asterisks indicate significantly different (*, P<0.005; **, P<0.001) allele ratios in the control and growth factor-treated xenografts. In the matched pair from donor #4 there was a decrease in the allele ratio for marker D3S1289 in the hyperplastic xenograft, which reached statistical significance when tested using the mean variance, but not when tested using the individual variance. C. Model for activation of the DNA damage checkpoint in cancer. Aberrant stimulation of cell proliferation, as occurs in preneoplastic lesions, leads to replication stress, DNA DSBs, genomic instability, activation of the DNA damage checkpoint and p53-dependent apoptosis. The latter suppresses expansion of the precancerous lesion (p53 tumor suppressor function) and provides selective pressure for p53 inactivation. Telomere attrition and hypoxia can also contribute to DNA DSB formation, activation of the checkpoint and genomic instability.

FIG. 4. Suppression of 53BP1 expression compromises p53-dependent apoptosis in tissue culture cells. A, Immunoblot analysis showing induction of ectopically-expressed p53 protein by doxycycline (Dox) and suppression of endogenous 53BP1 by siRNA in Saos2 osteosarcoma cells. B, Means and standard deviations of the percentages of apoptotic cells. ct1, control; bp1, 53 bp1. Methods: Saos2 osteosarcoma cells stably-expressing a tetracycline-inducible wild-type p53 gene 30 were transfected with control (luciferase) or 53 bp1-specific siRNA (ref. 15). 24 h later p53 expression was induced by doxycycline (800 ng/ml). The fraction of cells with sub-G1 DNA content was quantitated 48 h later by flow cytometry. The experiment was performed in triplicate.

FIG. 5. Immunohistochemistry of human normal skin (freckle) and a dysplastic nevus for Cdc2 phosphorylation on Tyr15.

FIG. 6. Immunohistochemistry of human foreskin xenografts for Cyclin E. Control, untreated xenograft; GFP, xenograft injected with adenovirus expressing GFP; GF, xenograft injected with adenoviruses expressing growth factors.

FIG. 7. The DNA double strand break (DSB) response in human cancer cell lines is associated with replication stress. A, H1stone H2AX phosphorylation in cancer cell lines occurs predominantly in S phase. Normal diploid human fibroblasts (DHF) and Saos2 osteosarcoma cells were arrested in G1 by serum starvation and doxycycline-dependent expression of p21/waf1, respectively, and then released into S phase. H1stone H2AX phosphorylation (γ-H2AX) and p21/waf1 (p21) protein levels were monitored by immunoblotting. The percentage of cells in S phase (% S) was determined by flow cytometry analysis. G1, population of cells arrested in G1; S, cells 20 h after release from the G1 arrest into S phase; A, asynchronous cell population. b, The γ-H2AX foci in non-irradiated Saos2 osteosarcoma and HeLa cervical carcinoma cells are associated with replication stress. Double immunofluorescence of non-irradiated (0 Gy) and irradiated (3 Gy, 30 min after irradiation) cells for γ-H2AX and ATRIP. Methods: Saos2 osteosarcoma cells stably-transfected with a doxycycline-inducible p21/waf1 gene were synchronized in G1 by inducing expression of p21/waf1 for 48 h with 800 ng/ml doxycyline and then released into S phase by doxycycline withdrawal and examined 20 h later. Diploid human lung fibroblasts (Gorgoulis et al., EMBO J. 22: 1567-1578, 2003) were arrested in G1 by serum withdrawal for 48 h and then released into S phase by adding media containing 10% fetal bovine serum.

FIG. 8. Comparative genomic hybridization (CGH) analysis of genomic DNA isolated from a lung hyperplasia. Positive findings would have been indicated by color-coded vertical lines adjacent to the chromosomes (red, loss; green, gain). Methods: CGH was performed as previously described (Levy et al., Genet. Med. 1: 4-12, 1998; Levy et al., Cancer Genet. Cytogenet. 121: 1-8, 2000). CGH profiles were compared to a dynamic standard reference interval based on the average of normal cases with the use of 95% confidence intervals (Kirchhoffet al., Cytometry 31: 163-173, 1998).

FIG. 9. Comparative genomic hybridization (CGH) analysis of genomic DNA isolated from a NSCLC (from the same patient, as shown in FIG. 8). Positive findings are indicated by color-coded vertical lines adjacent to the chromosomes (red, loss; green, gain).

### DETAILED DESCRIPTION

The present invention arises out of the surprising discovery of the identification of more than one marker of precancerous lesions and/or cancer. This invention can, in some embodiments, be used to detect the presence of precancerous lesions or cancer in a sample. In some embodiments, the invention can be used to distinguish precancerous hyperplastic and/or dysplastic lesions from non-precancerous tissue.

Cancer is defined as an abnormal mass of tissue, the growth of which exceeds and is uncoordinated with that of the normal tissues. Further, cancer has the potential of rapid growth, invasion, destruction of contiguous structures and dissemination throughout the body, leading to death.

The development of cancer requires several genetic changes (the precise number most likely varies from cancer to cancer). These genetic changes do not all occur at once. Accordingly, tumor progression is associated with the emergence of precursor lesions that exhibit only some of the properties of cancerous lesions. As used herein, the term “precancerous lesion” refers to these cancer-precursor lesions. Some precancerous lesions may acquire additional genetic changes and progress into cancer, while others may not progress to cancer in the lifetime of the patient, but nevertheless have genetic changes with oncogenic potential, such as mutations in proto-oncogenes and tumor suppressors. Most precancerous lesions exhibit growth exceeding that of the normal tissue, but all lack the ability to invade and metastasize.

The terms hyperplasia and dysplasia are often used to classify certain precancerous lesions. In hyperplasia there is an increase in the number of cells, these cells, however, maintain normal size, shape and organization. In dysplasia there is an alteration in cells characterized by variation in size, shape and organization. However, it should be noted that the terms hyperplasia and dysplasia do not apply only to precancerous lesions, but also to lesions induced by hormonal stimuli, chronic irritation and/or inflammation, because the terms hyperplasia and dysplasia are based on the morphology of a lesion, rather than on the presence of genetic changes with oncogenic potential.

Precancerous lesions can also be characterized by the presence of DNA replication stress. DNA replication stress is defined as a difficulty to properly initiate and/or complete DNA replication. For example, some origins for initiation of DNA replication may not fire (a term meaning to initiate DNA replication) or may fire more than once per cell cycle or may fire, even though they are not supposed to fire (not all origins of DNA replication normally fire during each cell cycle). Alternatively, DNA replication forks may be unable to properly complete DNA replication, because they stall and dissassemble into their individual subunits, requiring salvage pathways (such as, but not limited to, DNA repair pathways) to reinitiate DNA replication at the site of fork stalling and/or dissassembly).

DNA replication stress leads to phosphorylation of ATM and/or ATR substrates, as well as to loss of heterozygosity (LOH) at specific sites in the genome, called common fragile sites. Thus, the presence of phosphorylated ATM and/or ATR substrates in a tissue or LOH at common fragile sites in that tissue can distinguish precancerous tissue from normal tissue or hyperplastic/dysplastic non-precancerous tissue. In principle, any marker of DNA replication stress can be used for diagnosis of precancerous lesions. It is also evident that the presence of DNA replication stress in precancerous lesions could be exploited for therapy. Accordingly, in some embodiments, the present invention provides methods of determining the presence of and/or distinguishing precancerous hyperplastic and/or dysplastic lesions from non-precancerous tissue comprising determining the presence of DNA replication stress in the tissue, wherein the presence of DNA replication stress indicates the presence of a precancerous lesion.

In some embodiments, the present invention provides methods of detecting precancerous lesions and/or cancer comprising determining the phosphorylation status of a substrate of ATM and/or ATR in a sample, wherein the presence of a phosphorylated ATM and/or ATR substrate indicates the presence of a precancerous lesion and/or cancer. In some embodiments, the substrate of ATM and/or ATR is one that is phosphorylated in response to DNA damage. Examples of DNA damage include, but are not limited to, double-strand breaks, intra- and inter-strand crosslinks, and the like.

Whether or not a molecule is a substrate for ATM and/or ATR can be determined by one of skill in the art by any method. For example, a potential substrate can be contacted with ATM and/or ATR in vitro and the phosphorylation of the potential substrate can be measured. If the phosphorylation of the potential substrate is increased when contacted with ATM and/or ATR then the potential substrate is said to be a substrate of ATM and/or ATR. ATM substrates have a Ser-Gln or Thr-Gln motif, which is phosphorylated by ATM. Their phosphorylation is induced within 5-10 minutes after exposure to ionizing radiation in wild-type cells, but not in cells from patients in which the ATM gene is mutated (most ataxia-telangiectasia patients have mutations in the ATM gene). ATR substrates, most of which are also ATM substrates, also have a Ser-Gln or Thr-Gln motif, which is phosphorylated by ATR. Their phosphorylation is induced at later timepoints (2-6 hours) after exposure to ionizing radiation in wild-type cells, but is compromised in cells from patients with Seckel syndrome, in which the ATR gene is mutated. Once a substrate is known or identified by any method this substrate can be used in the present invention to detect the presence of precancerous lesions and/or cancer.

Lists of ATM and ATR substrates have been presented in the literature (for example, see Lavin et al., ATM and the DNA damage response: Workshop on Ataxia-Telangiectasia and Related Syndromes, EMBO Reports 7: 154-160, 2006). Examples of substrates of ATM and/or ATR include, but are not limited to Chk2, H2AX, SMC1, and the like. In some embodiments, the protein sequence of Chk2 comprises SEQ ID NO: 1 or a fragment thereof that is phosphorylated in response to DNA damage. In some embodiments, the protein sequence of H2AX comprises SEQ ID NO: 2 or a fragment thereof that is phosphorylated in response to DNA damage. A “fragment thereof that is phosphorylated in response to DNA damage” refers to the fragment of the protein that is phosphorylated when a cell's DNA is damaged.

In some embodiments, the determining the phosphorylation status of a substrate of ATM and/or ATR comprises contacting the sample with an agent to detect the phosphorylation of a substrate of ATM and/or ATR. In some embodiments, the agent is an antibody. In some embodiments, the antibody is a phospho-specific antibody. A “phospho-specific antibody” refers to an antibody that recognizes and binds to a phosphorylated residue and the binding of a phospho-specific antibody to a protein indicates that the protein is phosphorylated. In some embodiments, the antibody recognizes an ATM and/or ATR phosphorylation motif. An example of such a motif includes, but is not limited to X-Gln, wherein X is Ser or Thr. In some embodiments, the antibody or phospho-specific antibody, is a single chain antibody, monoclonal antibody, polyclonal antibody, Fab fragment of antibody, or a fragment of an antibody sufficient to bind to a substrate of ATM and/or ATR.

In some embodiments, the phosphorylation status of a substrate of ATM and/or ATR in the sample is compared to a normal sample, wherein an increase in phosphorylation as compared to the normal sample is indicative of the presence of a precancerous lesion and/or cancer. In some embodiments, the normal sample and the potentially non-normal sample can be isolated from one another or from the individual as a whole. Any method can be used including, but not limited to, microdissection.

The phosphorylation status of a substrate of ATM and/or ATR can also be determined by other methods including, for example, any immunohistochemistry method, immunoblotting, ELISA and mass spectrometery, that can be used to determine the phosphorylation status of the substrate. The exact method is not crucial but rather being able to determine the phosphorylation status of a substrate of ATM and/or ATR is what indicates the presence or absence of a precancerous lesion and/or cancer.

The substrate of ATM and/or ATR can be isolated from the sample and the phosphorylation status of the substrate can be determined on the isolated substrate. The isolated substrate can be compared to an isolated substrate from a normal tissue or cell, wherein an increase in phosphorylation on the substrate as compared to the normal tissue or cell indicates the presence of a precancerous lesion and/or cancer. Any method as described herein or used by one of skill in the art can be used to determine the phosphorylation status of a substrate of ATM and/or ATR.

In some embodiments, substrate of ATM and/or ATR is the checkpoint kinase Chk2. In some embodiments, the substrate is histone H2AX. In some embodiments, the method comprises determining the phosphorylation status of threonine 68 of Chk2. In some embodiments, the method comprises determining the phosphorylation status of serine 139 of H2AX. In some embodiments, the method comprises contacting a sample with a phospho-specific antibody to Chk2. In some embodiments, the method comprises contacting a sample with a phospho-specific antibody to H2AX. In some embodiments, the antibody binds to an phosphorylated threonine that corresponds to threonine 68 of Chk2. In some embodiments, the presence of a phosphorylated Chk2 indicates the presence of a precancerous lesion and/or cancer in the sample.

As used herein, the phrase “correspond to” a specific residue in a protein or “at a residue corresponding to” refers to a protein that comprises a residue that aligns with the specific residue described in the protein. Methods of determining alignment are well known in the art and any method can be used. For example, programs available in the Wisconsin Sequence Analysis Package, version 9.1 (Devereux J et al, Nucleic Acids Res, 12, 387-395, 1984, available from Genetics Computer Group, Madison, Wis., USA), can be used to determine the alignment of two proteins using programs such as, but not limited to, BESTFIT BESTFIT uses the “local homology” algorithm of Smith and Walerman (J Mol Biol, 147,195-197, 1981, Advances in Applied Mathematics, 2, 482-489, 1981) and finds the best single region of similarity between two sequences. BESTFIT is more suited to comparing two polynucleotide or two polypeptide sequences that are dissimilar in length, the program assuming that the shorter sequence represents a portion of the longer. The default settings for the alignment can be used. Other programs for determining identity and/or similarity between sequences are also known in the art, for instance the BLAST family of programs (Altschul S F et al, J Mol Biol, 215, 403-410, 1990, Altschul S F et al, Nucleic Acids Res., 25:389-3402, 1997, available from the National Center for Biotechnology Information (NCBI), Bethesda, Md., USA and accessible through the home page of the NCBI at www.ncbi.nim.nih.gov) and FASTA (Pearson W R, Methods in Enzymology, 183, 63-99, 1990; Pearson W R and Lipman D J, Proc Nat Acad Sci USA, 85, 2444-2448, 1988, available as part of the Wisconsin Sequence Analysis Package). In some embodiments, the BLOSUM62 amino acid substitution matrix (Henikoff S and Henikoff J G, Proc. Nat. Acad. Sci. USA, 89, 10915-10919, 1992) is used in polypeptide sequence comparisons including, but not limited to, where nucleotide sequences are first translated into amino acid sequences before comparison.

In some embodiments, the protein that is detected is H2AX. H2AX phosphorylated on Ser139 can also be referred to as γ-H2AX. In some embodiments, a binding agent (e.g. an antibody) will recognized a phosphorylated H2AX. In some embodiments H2AX is phosphorylated within a Ser-Gln motif. In some embodiments, the phosphorylation occurs at Ser 139 of H2AX or a position that corresponds to Ser 139 of H2AX. In some embodiments, the presence of a phosphorylated H2AX indicates the presence of a precancerous lesion and/or cancer in the sample.

In some embodiments, the present invention provides methods of detecting a precancerous lesion and/or cancer in a subject, the method comprising: detecting the phosphorylation status of SMC1, Chk2 and/or H2AX in a test sample obtained from a subject; and comparing the phosphorylation status of SMC1, Chk2 and/or H2AX in test sample to a level of phosphorylation in a normal, non-cancer or non-precancerous cell. In some embodiments, the normal cell is of the same tissue type as the suspected cancer cell. In some embodiments, an increase in SMC1 phosphorylation, Chk2 phosphorylation or H2AX phosphorylation indicates that the subject has a precancerous lesion and/or cancer.

In some embodiments, the present invention provides methods for identifying a subject at risk of cancer, which comprises detecting the presence or absence phosphorylated substrate of ATM and/or ATR, such as, but not limited to, SMC1, Chk2 and/or H2AX, wherein the presence of the phosphorylation of SMC1, Chk2 and/or H2AX is indicative of the subject being at risk of cancer. In some embodiments, the phosphorylation of Chk2 is at position corresponding to Threonine-68 of Chk2. In some embodiments, the phosphorylation of H2AX is at a position corresponding to or at position 139 of H2AX.

In some embodiments, the present invention provides methods of detecting precancerous lesions and/or cancer comprising contacting an agent with a test sample and detecting whether the binding entity binds to an epitope comprising Ser-Gln or Thr-Gln or a phosphorylated epitope comprising Ser-Gln or Thr-Gln, wherein if the agent detects and binds a phosphorylated epitope comprising Ser-Gln or Thr-Gln indicates that the sample is a precancerous lesion and/or cancer.

As used herein the term “sample” can be taken from an individual (human) or a non-human animal (e.g. mouse, rat, cat, dog, and the like). The sample can be any in any form, such as but not limited to a tissue sample, cell isolation, and the like. In some embodiments, the cells in the sample are fixed. In some embodiments, the cells are not fixed. The samples can be prepared in such a way to make a cellular extract. The cellular extract can be used to isolate a substrate of ATM and/or ATR or to detect the presence and/or the phosphorylation status of a substrate of ATM or/ATR. The tissue sample can be from any tissue including, but not limited to breast, pancreas, lung, muscle, brain, ovarian, stomach, colon, esophageal cells, lymph node and the like.

The sample being tested can be from an individual or non-human animal suspected of having cancer or precancerous lesion or from an individual or non-human animal not suspected of having cancer or precancerous lesion or no determination at all has been made as to whether the person may or may not have cancer or a precancerous lesion.

The present invention also provides methods of detecting a precancerous lesion comprising detecting in a sample loss of heterozygosity (LOH) at a common fragile site wherein a LOH at the common fragile site indicates the presence of a precancerous lesion.

As used herein the term “common fragile site” refers to sites in the genome that have a higher likelihood than other sites in the genome of showing site-specific gaps and breaks on metaphase chromosomes after partial inhibition of DNA synthesis. Eleven common fragile sites have been cloned in the human genome (for a list of these sites see Table 1 of Glover TW. Common fragile sites. Cancer Letters 232: 4-12, 2006). An example is the FRA3B common fragile site on the short arm of chromosome 3. Additional common fragile sites are likely to be discovered.

LOH can be detected by any method including microarrays (see, for example Bartkova Nature, Vol 434, 864-870 (2005)), PCR (see Example 1, below), and the like. The method can include the detection of single nucleotide polymorphisms (SNP) to detect allelic imbalance affecting the common fragile site to determine if there has been a loss of heterozygosity. The method can also include the detection of polymorphic microsatellite markers to detect allelic imbalance affecting the common fragile site. In some embodiments, the LOH in the sample is compared to the LOH status in a normal sample.

In some embodiments, the present invention provides methods of detecting precancerous lesions comprising detecting loss of heterozygosity (LOH) at the FRA3B common fragile site. In some embodiments, LOH is detected or monitored by using the markers D3S1289 and/or D3S1300. However, other markers can also be used to measure LOH at the FRA3B site. In some embodiments, the LOH at this site is monitored in suspected tissue and compared to normal tissue. Any normal tissue can be used such as, but not limited to, peripheral blood lymphocytes. In some embodiments, the LOH at the FRA3B site indicates that the sample is precancerous and/or cancerous.

The present invention also provides for kits for detecting the presence of a precancerous lesion and/or cancer, as described herein. In some embodiments, the kit comprises at least one antibody that can detect Chk2, SMC1, and/or H2AX. In some embodiments, the kit comprises a phospho-specific antibody. The kit can also provide reagents for detecting LOH at common fragile sites. In some embodiments, the kit comprises primers for detecting LOH at common fragile sites. In some embodiments, the kit comprises primers for detecting LOH at FRA3B. In some embodiments, the primers are to the markers D3S1289 and/or D3S1300. Analysis of LOH at control sites in the genome (i.e. sites that are not common fragile sites) can be used as a control. In precancerous lesions the LOH affects preferentially the common fragile sites.

The invention is now described with reference to the following examples. These examples are provided for the purpose of illustration only and the invention should in no way be construed as being limited to these examples, but rather should be construed to encompass any and all variations which become evident as a result of the teaching provided herein. Those of skill in the art will readily recognize a variety of non-critical parameters that could be changed or modified to yield essentially similar results.

### EXAMPLES

### Example 1

In human cancer, DNA damage checkpoint genes, such as p53, are frequently mutated, but the selective pressure for their inactivation remains elusive. A panel of human lung precancerous hyperplasias was analyzed. All of these precancerous hyperplasias retained wild-type p53 genes and had no signs of gross chromosomal instability. Yet, all these hyperplasias had signs of a DNA damage response, including histone H2AX and Chk2 phosphorylation, 53BP1 focal staining, p53 accumulation and apoptosis. Progression to carcinoma (cancer) was associated with p53 or 53BP1 inactivation and decreased apoptosis. A DNA damage response was also observed in precancerous dysplastic nevi and in human skin xenografts, in which hyperplasia resembling human precancerous lesions was induced by overexpressing growth factors. Both the lung and experimentally-induced skin hyperplasias exhibited allelic imbalance at loci that are prone to DNA double strand break (DSB) formation when DNA replication is compromised (common fragile sites).

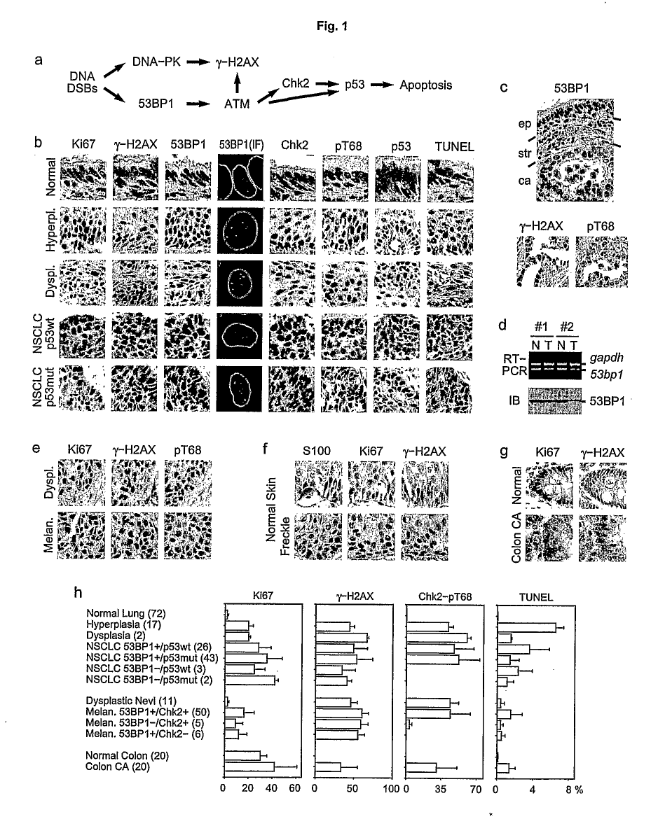

The most frequently mutated gene in human cancer is p53, a gene that functions in the checkpoint response to DNA DSBs (FIG. 1a; refs 1, 2). Several models, not mutually exclusive with each other, have been proposed to explain the high frequency of p53 inactivation³⁻⁶. One of the prevailing models states that tumor growth leads to telomere attrition and hypoxia resulting in a DNA damage response⁴⁻⁶. This model predicts that the DNA damage response occurs some time after cancer initiation³. Here, a systematic analysis of precancerous and cancer lesions was performed to determine how early during human cancer development a DNA DSB checkpoint response might become evident.

A previously described panel of surgically-resected non-small cell lung carcinomas (NSCLCs) from patients who had received no form of cancer therapy prior to surgery^{7,8} were examined. Almost all specimens in this panel (N=74) contained normal adjacent lung tissue (N=72) and some also contained hyperplastic (N=17) and dysplastic lesions (N=2), whose location suggested that they were precancerous lesions and precursors of the adjacent NSCLCs. The p53 gene was wild-type in all the precancerous hyperplasias, mutant in the precancerous dysplasias and either mutant (N=45) or wild-type (N=29) in the NSCLCs. In the two dysplasias the p53 mutations were the same as in the adjacent NSCLCs, consistent with the precancerous dysplasias being precursors of the adjacent NSCLCs (data not shown).

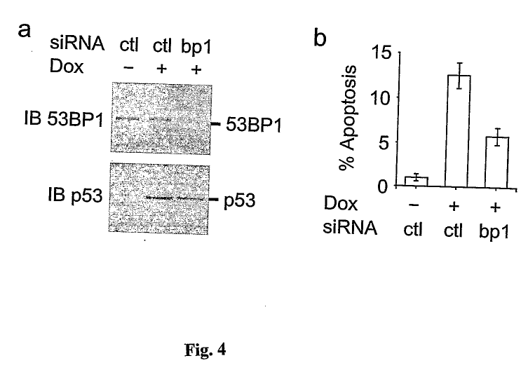

The presence of a DNA damage response can be ascertained by monitoring histone H2AX phosphorylation (γ-H2AX), 53BP1 intracellular localization, Chk2 phosphorylation on Thr68 and p53 protein levels (FIG. 1a; refs 9-13). In the normal lung epithelium all markers were consistent with the absence of a DNA damage response (FIG. 1b). However, in the hyperplasias there were signs that the DNA DSB checkpoint pathway had been activated. 53BP1, which is a sensor of DNA DSBs (ref. 14), localized to discrete nuclear foci, reminiscent of the 53BP1 foci that are observed in irradiated tissue culture cells and which represent sites of DNA DSBs². In addition, histone H2AX and Chk2 were phosphorylated; p53 protein levels were elevated and some cells were undergoing apoptosis (FIG. 1b). In the dysplasias and NSCLCs there were also signs of a DNA damage response as evidenced by 53BP1 localization and histone H2AX and Chk2 phosphorylation. However, apoptosis was suppressed relative to the hyperplasias. In most cases this was associated with p53 mutations or with low levels of wild-type p53 protein (FIG. 1b, h). In a small number of NSCLCs decreased apoptosis was associated with low levels of 53BP1 mRNA and protein (FIG. 1c, d, h). In these NSCLCs, Chk2 was not phosphorylated on Thr68, whereas histone H2AX phosphorylation persisted (FIG. 1c, h). These effects of lack of 53BP1 expression in NSCLCs fully recapitulate the effects of 53BP1 depletion in tissue culture cells (FIG. 4 and refs 13, 15).

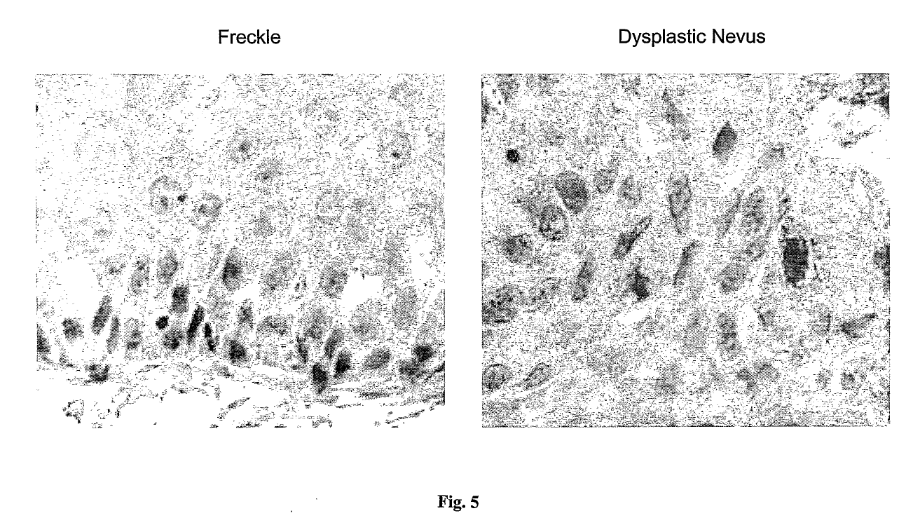

To expand these studies to other tumor types a cohort of patients with malignant melanoma (N=61) was examined. Eleven of these patients also had precancerous dysplastic nevi adjacent to their melanoma. All the dysplastic nevi, most of which were from areas of the body not exposed to sunlight, stained positive for phosphorylated histone H2AX and Chk2 (FIG. 1e, h), whereas the adjacent skin always stained negative (FIG. 1f). The melanomas also stained positive for phosphorylated histone H2AX and Chk2, with the exception of a small number of melanomas, which stained positive for γ-H2AX, but negative for phosphorylated Chk2. These latter melanomas did not express either 53BP1 (N=5) or Chk2 (N=6) protein (FIG. 1h). Unlike the lung precancerous hyperplasias, the frequency of apoptotic cells in the precancerous dysplastic nevi was low and did not decrease during progression to melanoma. Instead, progression to melanoma was associated with an increase in the proliferation index (FIG. 1h). Although several models can explain the increased proliferation that accompanies progression from dysplastic nevi to melanoma, one possibility is that activation of the DNA damage checkpoint in dysplastic nevi is inducing cell cycle arrest and that progression to melanoma is associated with escape from this arrest through various mechanisms, including suppression of 53BP1 and Chk2 expression. In support of this model, dysplastic nevi stained positive for Cdc2 phosphorylated on Tyr15 (FIG. 5), a marker of activation of the G2 checkpoint^{2,11}.

The results presented above, as well as similar and complementary findings from Jiri Bartek's group¹⁶, suggest that the DNA damage checkpoint is activated in a wide variety of human preneoplastic and neoplastic lesions. Alternatively, it is formally possible that the markers studied above score positive during normal cell proliferation, in which case the response of the preneoplastic and neoplastic lesions could simply be a reflection of their high proliferation index (FIG. 1h). To explore this possibility normal colonic crypts in surgically resected tissues from colon cancer patients were examined. Despite having a higher proliferation index than the lung and melanocytic preneoplastic and neoplastic lesions, the normal colonic epithelium (N=20) stained uniformly negative for both histone H2AX and Chk2 phosphorylation (FIG. 1g, h). Normal skin, which has a relatively high proliferation index, also stained negative (FIG. 1f and data not shown). Thus, activation of the DNA damage checkpoint occurs specifically in preneoplastic and neoplastic lesions.

The precancerous lung hyperplasias and dysplastic nevi that were studied are very early lesions in terms of stage of cancer development. However, since cancer probably takes years to develop, it is possible that when these precancerous lesions first formed they did not exhibit a DNA damage response. To address this issue a precancerous hyperplasia model¹⁷ was studied, in which human skin xenografts were implanted on the backs of immunodeficient mice and then induced to become hyperplastic by 4 weekly subcutaneous injections of adenoviral vectors expressing growth factors (basic fibroblast growth factor, stem cell factor and endothelin-3). Control xenografts were either not injected or injected with an adenovirus that expresses green fluorescent protein (GFP). Both newborn foreskin and adult skin (from breast reduction cosmetic surgeries) were grafted. Foreskin from a specific donor, due to its small size, was grafted on a single mouse, such that the various treatment groups comprised grafts from different individuals. The larger adult skin samples were used to prepare 2 grafts; one was injected with adenoviruses expressing growth factors, while the other served as a donor-matched untreated control. All grafts were harvested for analysis one week after the last injection.

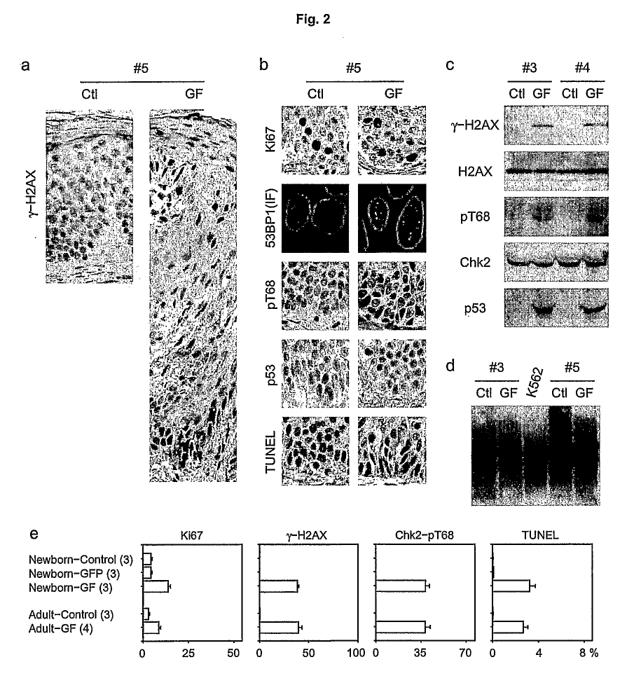

Both the foreskin and adult skin-derived hyperplastic xenografts fully recapitulated the lung hyperplasias in terms of DNA damage response: 53BP1 localized at discrete nuclear foci, histone H2AX and Chk2 were phosphorylated, p53 protein levels were induced and there was apoptosis (FIG. 2). None of these effects were observed in the non-injected controls or the controls injected with the adenovirus expressing GFP (FIG. 2e).

One possible mechanism for activation of the DNA damage checkpoint in the precancerous human lesions and hyperplastic skin xenografts might involve telomere attrition. Therefore, telomere lengths in matched control and hyperplastic adult skin xenograft pairs were compared, but observed no differences, whereas shortened telomeres were readily evident in the K562 erythroleukemia cancer cell line (FIG. 2d). The absence of telomere attrition, at least at this level of analysis, is perhaps not surprising given that the xenografts were obtained from newborns and young adults and were examined just a few weeks after grafting.

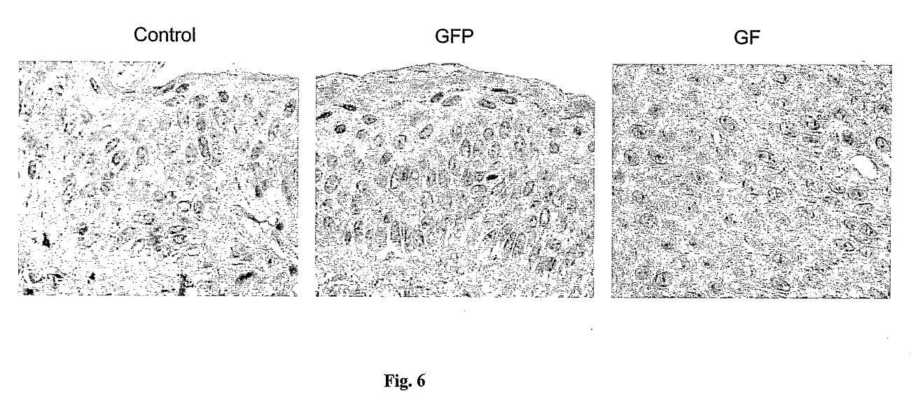

Another possible mechanism for the DNA damage response observed in the precancerous lesions might involve replication stress. High levels of Cyclin E protein were noted in both the precancerous lung hyperplasias and hyperplastic human skin xenografts (FIG. 6 and data not shown). High levels of Cyclin E and more broadly deregulation of cyclin-dependent kinase activity in G1 are very frequent in human cancer and compromise prereplication complex assembly and licensing of origins of replication. As a result, too few origins may fire or some origins may fire more than once per cell cycle leading to replication stress, which in turn could lead to DNA DSBs, activation of the DNA damage checkpoint and even genomic instability¹⁸⁻²².

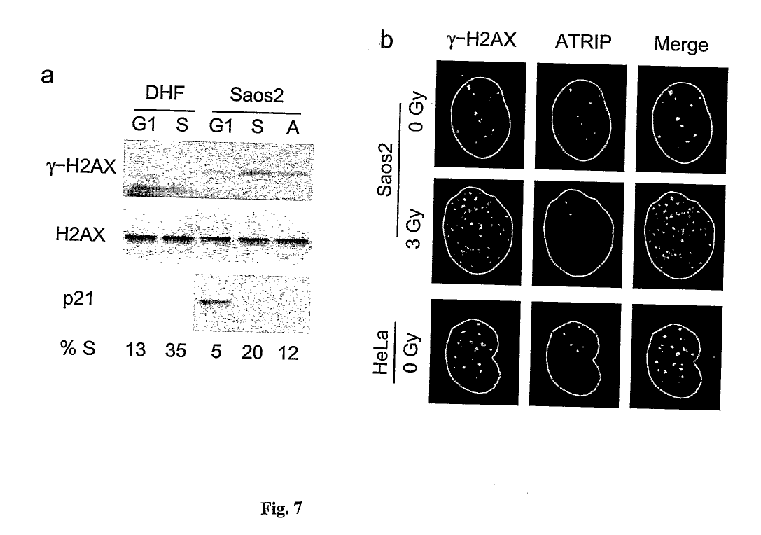

As a first step in linking replication stress to the DNA damage response observed in precancerous lesions and in cancer, cancer cell lines, such as Saos2 osteosarcoma and HeLa cervical carcinoma, were studied in which the DNA damage checkpoint is active even in the absence of exposure to ionizing radiation¹⁵. If the DNA damage response in these cell lines is due to DNA replication stress, it should be dependent on entry into S phase. Further, the γ-H2AX foci in the nuclei of these cells should colocalize with ATRIP-ATR foci, since the ATRIP-ATR complex is recruited to sites of DNA replication stress^{24,25}. Both predictions were true. Saos2 cells stably-transfected with a doxycycline-inducible p21/waf1 gene were synchronized in G1 by inducing expression of p21/waf1 and then released into S phase by doxycycline withdrawal. H1stone H2AX phosphorylation was more robust in the cycling cells than in the cells arrested in G1, whereas in early passage human diploid lung fibroblasts histone H2AX was not phosphorylated, irrespective of whether the cells were cycling or resting (FIG. 7a and ref. 23). Further, by immunofluorescence a majority of γ-H2AX foci in non-irradiated Saos2 and HeLa cells colocalized with ATRIP foci; whereas, as a control, γ-H2AX foci induced in these cell lines in response to ionizing radiation were devoid of ATRIP (FIG. 7b).

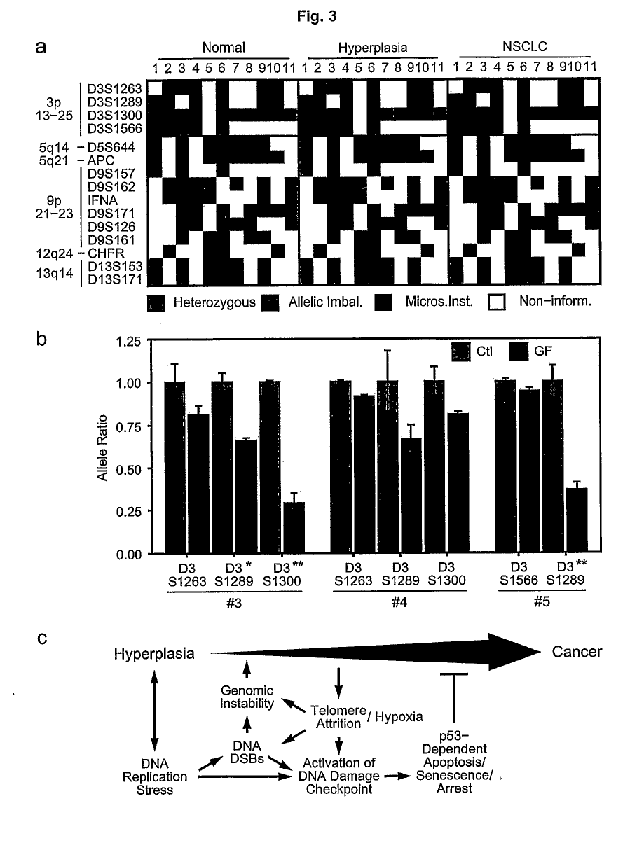

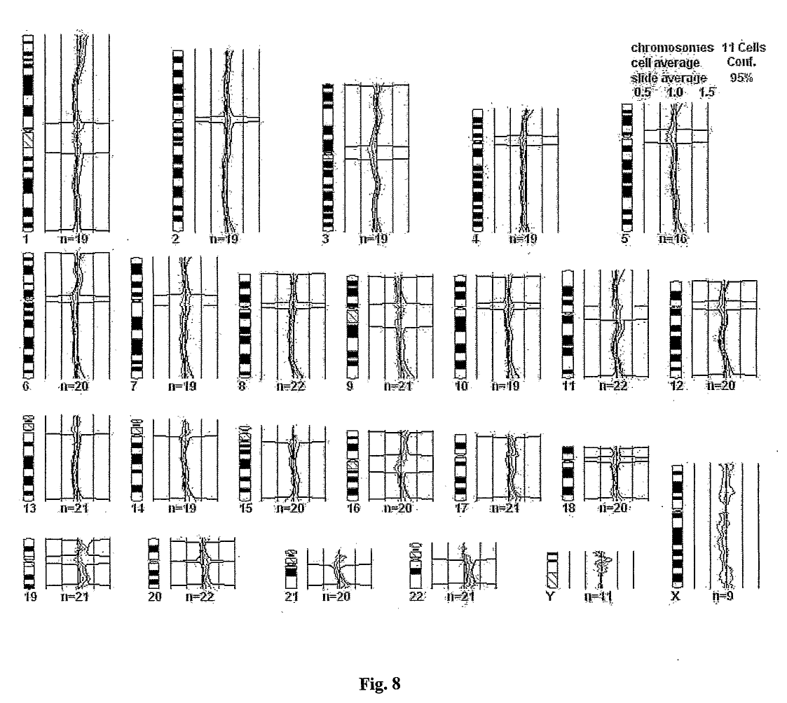

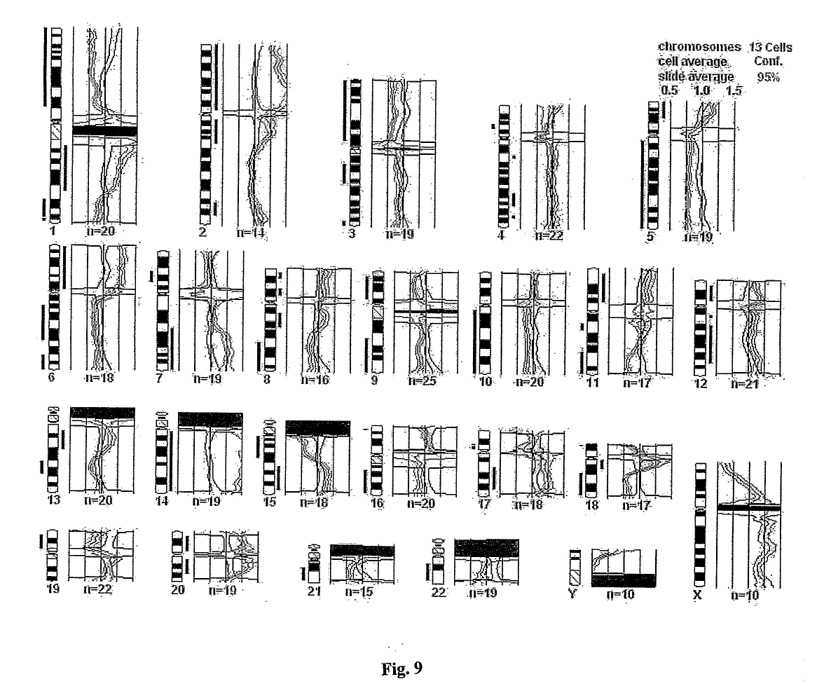

To generate evidence for the presence of DNA replication stress in preneoplastic human lesions it was reasoned that DNA replication stress through formation of DNA DSBs and defective DNA repair could lead to allelic imbalances. The loci in the genome that are prone to DNA DSB formation in the presence of DNA replication stress are called common fragile sites^{26,27}. Therefore, it was predicted that these sites would be preferentially targeted for allelic imbalance in human preneoplastic lesions and tested this hypothesis using genomic DNA isolated from normal bronchial epithelium, precancerous hyperplastic tissue and cancer tissue from 11 NSCLC patients. Allelic imbalance at the most common fragile site (FRA3B on chromosome 3p14) was compared to loci on chromosomes that commonly exhibit allelic imbalance in advanced human cancers, but which do not correspond to common fragile sites. In the precancerous hyperplasias allelic imbalance affecting the common fragile site FRA3B was very frequent (and occasionally extended to more distant loci on 3p), whereas the other chromosomal loci were either not affected or minimally affected (FIG. 3a). Consistent with preferential targeting of common fragile sites in these hyperplasias, comparative genomic hybridization analysis also revealed absence of gross chromosomal instability (FIG. 8 and data not shown). In the NSCLCs the allelic imbalances established in the precancerous hyperplastic stage persisted (providing evidence for progression from hyperplasia to NSCLC), but additional allelic imbalances, affecting known tumor suppressor loci, and gross chromosomal instability were also present (FIG. 3a, FIG. 9 and data not shown). These, and similar results by others^{16,28-30}, are consistent with the presence of DNA replication stress in human preneoplastic lesions.

The allelic imbalance analysis of common fragile sites was extended to the matched pairs of control and hyperplastic adult skin xenografts. Genomic DNA was isolated from 2-5 serial sections of xenograft tissue and allelic imbalance was examined using 2 microsatellite markers (D3S1289 and D3S1300) that map to the FRA3B common fragile site on chromosome 3p and 2 markers (D3S1263 and D3S1566) that map to non-fragile sites on chromosome 3p (3p25 and 3 p13, respectively). In all 3 matched pairs examined, allelic imbalance at FRA3B was evident in the hyperplastic xenograft, whereas the 3p25 and 3 p13 loci remained heterozygous (FIG. 3b). Thus, in an experimental human precancerous model, aberrant growth factor signaling associated with loss of tissue homeostasis (precancerous hyperplasia) can lead within a few weeks to allelic imbalance at common fragile sites.

These findings suggest a model that may explain the tumor suppressor function of p53 and also hints to mechanisms leading to genomic instability in early cancer lesions (FIG. 3c). Specifically, it is proposed that from the very beginning of cancer development, deregulation of proto-oncogenes and/or tumor suppressors that promotes aberrant cell proliferation leads to replication stress. The latter, either directly or through formation of DNA DSBs, can activate the DNA damage checkpoint, which thus, functions as a tumor suppressor by inducing cell cycle arrest or apoptosis. In those cells that do not undergo permanent cell cycle arrest or apoptosis, there is a likelihood of errors in DNA DSB repair leading to allelic imbalances. These imbalances will target preferentially the common fragile sites, since these sites are most sensitive to DNA replication stress. At later stages, telomere attrition and hypoxia will also contribute to checkpoint activation and genomic instability⁴⁻⁶. Eventually, tumor suppressor loci, such as p53, will be targeted, releasing the cells from the suppressor effects of the DNA damage checkpoint pathway and facilitating tumor progression.

### Example 2

### Methods

### Antibodies

For immunohistochemistry, immunofluorescence and immunoblot analysis previously-characterized primary antibodies¹³⁻¹⁵ were used at the dilutions indicated below: anti-phospho-H2AX (Ser 39) (1:100; Upstate); anti-53BP1 and anti-Chk2 (hybridoma supernatants 1:20; ref. 13-15); anti-phospho-Chk2 (Thr68, Lot 1) (1:100; Cell Signaling Technology); anti-p53 (DO7) (1:100; Dako); anti-Ki67 (MIB-1) (1:100, Dako); and anti-S100 (1:100, Dako).

Tissue Samples

A database of frozen and formalin-fixed paraffin-embedded material from a total of 74 resected NSCLCs, adjacent normal lung tissue and corresponding precancerous lesions (17 cases of hyperplasias/metaplasias with two cases bearing also dysplasias) has been described^{7,8}. 61 cases of sporadic malignant melanoma, 11 of which developed from dysplastic nevi, and 20 non-familial colon carcinoma cases were selected without bias from the patient population of the “Agios Savas” Hospital in Athens, Greece. None of the patients had undergone cancer therapy prior to surgical resection of the lesions.

Human Skin Xenograft Model

The human skin xenograft model has been described¹⁷. For these studies 9 newborn foreskin xenografts were examined: 3 were injected subcutaneously on a weekly basis over a period of 4 weeks with adenoviruses expressing basic fibroblast growth factor, stem cell factor and endothelin-3; 3 with an adenovirus expressing GFP; and 3 were untreated. Four adult skin grafts from patients undergoing breast reduction cosmetic surgeries were also studied. These grafts were cut in half and each half was implanted in separate SCID mice. In one mouse the graft was injected with adenoviral vectors expressing the growth factors listed above; in the other mouse the graft was untreated. All grafts were harvested a week after the last injection. The foreskin grafts were fixed with formalin and analyzed by immunohistochemistry. Part of the adult skin grafts was flash-frozen and used to prepare protein extracts, while another part was fixed with formalin and analyzed by immunohistochemistry or used to prepare genomic DNA. One of the adult untreated grafts was damaged during sectioning and was not analyzed by immunohistochemistry.

Analysis of Tissue Samples

Formalin-fixed tissue sections were processed for immunohistochemistry, immunofluorescence and Tdt-mediated dUTP nick-end labeling (TUNEL) analysis as previously described^{7,8}. RNA was extracted from frozen samples and used to prepare cDNA (ref. 7,8). For analysis of 53 bp1 expression at the mRNA level, the cDNA was amplified by semi-quantitative multiplex polymerase chain reaction (PCR) using primers specific for 53 bp1 (GCAGCCTCTGTGAAGCAGCA and ATGCAAGGAATCCAGTTACACACAA) and gapdh, as standard^{7,8}. p53 mutations were identified by single strand conformation polymorphism analysis and sequencing^{7,8}. Proteins were extracted by lysis of minced frozen samples in RIPA buffer supplemented with protease inhibitors¹³. Histones were isolated from the RIPA-insoluble pellet by extraction with buffer consisting of 10 mM HEPES, 1.5 mM MgCl₂, 10 mM KCI, 0.5 mM DTT, 1.5 mM PMSF and 0.25 N HCI for 1 h at 4° C. Immunoblotting was performed as previously described¹³.

Telomere Length Assay

Telomere lengths were determined using the TeloTAGGG Telomere Length Assay (Roche Diagnostics) according to manufacturer's instructions. Briefly, 4 μg genomic DNA isolated from 2 matched pairs of control and growth factor-treated adult skin xenografts was digested with Hinfl and RsaI and subjected to Southern blot analysis using telomere-specific labeled probes. DNA prepared from the K562 erythroleukemic cell line served as a control.

Allelic Imbalance Analysis

Allelic imbalance analysis of lung tissues was tabulated from a previously published studies (ref. 7,8 and references therein) or extended to include microsatellite markers for chromosome 3p. For analysis of human skin xenografts, 2-5 serial 10μ-thick paraffin-embedded sections were microdissected using laser capture and genomic DNA was extracted as previously described⁷. Each genomic DNA sample was subjected to 2 independent polymerase chain reactions (PCR) and the PCR products were resolved using a 377 ABI PRISM automated sequencer (Applied Biosystems), as previously described⁸. Differences in allele ratios between control and growth factor-treated xenografts were evaluated twice for statistical significance using the individual variances and the mean variance calculated from all the replicates and were scored positive only if both evaluations showed statistical significance.

### REFERENCES

1. Hollstein, M., Sidransky, D., Vogelstein, B. & Harris, C. C. p53 mutations in human cancers. Science 253, 49-53 (1991).
2. Kastan, M. B. & Bartek, J. Cell-cycle checkpoints and cancer. Nature 432, 316-323 (2004).
3. Halazonetis, T. D. Constitutively active DNA damage checkpoint pathways as the driving force for the high frequency of p53 mutations in human cancer. DNA Repair 3, 1057-1062 (2004).
4. Takai, H., Smogorzewska, A. & de Lange, T. DNA damage foci at dysfunctional telomeres. Curr. Biol. 13, 1549-1556 (2003).
5. d'Adda di Fagagna, F. et al. A DNA damage checkpoint response in telomere-initiated senescence. Nature 426, 194-198 (2003).
6. Graeber, T. G. et al. Hypoxia-mediated selection of cells with diminished apoptotic potential in solid tumours. Nature 379, 88-91 (1996).
7. Gorgoulis, V. G. et al. Alterations of the p16-pRb pathway and the chromosome locus 9p21-22 in non-small-cell lung carcinomas: relationship with p53 and MDM2 protein expression. Am. J. Pathol. 153, 1749-1765 (1998).
8. Karakaidos, P. et al. Overexpression of the replication licensing regulators hCdt1 and hCdc6 characterizes a subset of non-small-cell lung carcinomas: synergistic effect with mutant p53 on tumor growth and chromosomal instability—evidence of E2F-1 transcriptional control over hCdt1. Am. J. Pathol. 165, 1351-1365 (2004).
9. Kastan, M. B., Onyekwere, O., Sidransky, D., Vogelstein, B. & Craig, R. W. Participation of p53 protein in the cellular response to DNA damage. Cancer Res. 51, 6304-6311 (1991).
10. Rogakou, E. P., Pilch, D. R., Orr, A. H., Ivanova, V. S. & Bonner, W. M. DNA double-stranded breaks induce histone H2AX phosphorylation on serine 139. J. Biol. Chem. 273, 5858-5868 (1998).
11. Bartek, J. & Lukas, J. Chk1 and Chk2 kinases in checkpoint control and cancer. Cancer Cell 3, 421-429 (2003).
12. Schultz, L. B., Chehab, N. H., Malikzay, A. & Halazonetis, T. D. p53 binding protein 1 (53BP1) is an early participant in the cellular response to DNA double-strand breaks. J Cell Biol. 151, 1381-1390 (2000).
13. Mochan, T. A., Venere, M., DiTullio, R. A., Jr. & Halazonetis, T. D. 53BP1 and NFBD1/MDC1-Nbs1 function in parallel interacting pathways activating ataxia-telangiectasia mutated (ATM) in response to DNA damage. Cancer Res. 63, 8586-8591 (2003).
14. Huyen, Y. et al. Methylated lysine 79 of histone H3 targets 53BP1 to DNA double-strand breaks. Nature 432, 406-411 (2004).
15. DiTullio, R. A., Jr. et al. 53BP1 functions in an ATM-dependent checkpoint pathway that is constitutively activated in human cancer. Nat. Cell Biol. 4, 998-1002 (2002).
16. Bartkova, J. et al. DNA damage response as a candidate anti-cancer barrier in early human tumourigenesis. Nature submitted manuscript (2005).
17. Berking, C. et al. Induction of melanoma phenotypes in human skin by growth factors and ultraviolet B. Cancer Res. 64, 807-811 (2004).
18. Spruck, C. H., Won, K. A. & Reed, S. I. Deregulated cyclin E induces chromosome instability. Nature 401, 297-300 (1999).
19. Lengronne, A. & Schwob, E. The yeast CDK inhibitor Sic1 prevents genomic instability by promoting replication origin licensing in late G1. Mol. Cell 9, 1067-1078 (2002).
20. Tanaka, S. & Diffley, J. F. Deregulated G1-cyclin expression induces genomic instability by preventing efficient pre-RC formation. Genes Dev. 16, 2639-2649 (2002).
21. Vaziri, C. et al. A p53-dependent checkpoint pathway prevents rereplication. Mol Cell 11, 997-1008 (2003).
22. Ekholm-Reed, S., Mendez, J., Tedesco, D., Zetterberg, A., Stillman, B. & Reed, S. I. Deregulation of cyclin E in human cells interferes with prereplication complex assembly. J. Cell Biol. 165, 789-800 (2004).
23. MacPhail, S. H., Banath, J. P., Yu, Y., Chu, E. & Olive, P. L. Cell cycle-dependent expression of phosphorylated histone H2AX: reduced expression in unirradiated but not X-irradiated G1-phase cells. Radiat. Res. 159, 759-767 (2003).
24. Tibbetts, R. S. et al. Functional interactions between BRCA1 and the checkpoint kinase ATR during genotoxic stress. Genes Dev. 14, 2989-3002 (2000).
25. Cortez, D., Guntuku, S., Qin, J. & Elledge, S. J. ATR and ATRIP: partners in checkpoint signaling. Science 294, 1713-1716 (2001).
26. Arlt, M. F., Casper, A. M. & Glover, T. W. Common fragile sites. Cytogenet. Genome Res. 100, 92-100 (2003).
27. Casper, A. M., Nghiem, P., Arlt, M. F. & Glover, T. W. ATR regulates fragile site stability. Cell 111, 779-789 (2002).
28. Mao, L. et al. Frequent microsatellite alterations at chromosomes 9p21 and 3 p14 in oral premalignant lesions and their value in cancer risk assessment. Nat. Med. 2, 682-685 (1996).
29. Wistuba, I. I. et al. High resolution chromosome 3p allelotyping of human lung cancer and preneoplastic/preinvasive bronchial epithelium reveals multiple, discontinuous sites of 3p allele loss and three regions of frequent breakpoints. Cancer Res. 60, 1949-1960 (2000).
30. Maitra, A. et al. High-resolution chromosome 3p allelotyping of breast carcinomas and precursor lesions demonstrates frequent loss of heterozygosity and a discontinuous pattern of allele loss. Am. J. Pathol. 159, 119-103 (2001).

The disclosures of each and every patent, patent application, publication, reference and accession number cited herein are hereby incorporated herein by reference in their entirety.

1. A method of determining the presence of precancerous hyperplastic and/or dysplastic lesions and/or distinguishing precancerous hyperplastic and/or dysplastic lesions from non-precancerous tissue comprising determining the phosphorylation status of a substrate of ATM and/or ATR, wherein the presence of a phosphorylated ATM and/or ATR substrate indicates the presence of a precancerous lesion.

2. The method of claim 1 wherein said determining comprises contacting said sample with an agent to detect the phosphorylation of a substrate of ATM and/or ATR.

3. The method of claim 1 wherein said substrate is Chk2, H2AX, or SMC1.

4. The method of claim 3 wherein the phosphorylation of Chk2 is detected at a residue corresponding to threonine 68 of SEQ ID NO: 1.

5. The method of claim 3 wherein the phosphorylation of H2AX is detected at a residue corresponding to serine 139 of SEQ ID NO: 2.

6. The method of claim 1 wherein the phosphorylation status of a substrate of ATM and/or ATR in the sample is compared to a normal sample, wherein an increase in phosphorylation as compared to the normal sample is indicative of the presence of a precancerous lesion.

7. (canceled)

8. The method of claim 2 wherein said agent is an antibody.

9. (canceled)

10. The method of claim 1 wherein said determining comprises isolating said substrate of ATM and/or ATR from said sample and detecting phosphorylation of said substrate of ATM and/or ATR.

11. (canceled)

12. The method of claim 10 wherein said detecting phosphorylation comprises contacting said isolated substrate of ATM and/or ATR with an agent to detect phosphorylation.

13. The method of claim 12 wherein said agent is an antibody.

14. (canceled)

15. The method of claim 13 wherein said antibody is an antibody that recognizes X-GIn motif, wherein X is Ser or Thr.

16. The method of claim 1 wherein said sample is a sample taken from an individual.

17.-19. (canceled)

20. The method of claim 16 wherein said individual is suspected of having cancer.

21. A method of determining the presence of precancerous hyperplastic and/or dysplastic lesions and/or distinguishing precancerous hyperplastic and/or dysplastic lesions from non-precancerous tissue comprising determining the presence of DNA replication stress in the tissue, wherein the presence of DNA replication stress indicates the presence of a precancerous lesion.

22. A method of detecting the presence of and distinguishing precancerous hyperplastic and/or dysplastic lesions from non-precancerous tissue comprising detecting in a sample loss of heterozygosity (LOH) at a common fragile site wherein a LOH at the common fragile site indicates the presence of a precancerous lesion.

23. The method of claim 22 wherein said fragile site is the FRA3B common fragile site.

24. The method of claim 22, wherein LOH is detected using the microsatellite markers D3S1289 and/or D3S1300, wherein a change in the allele ratio compared to normal tissue from the same patient indicates LOH and the presence of a precancerous lesion.

25. The method of claim 22 wherein said detecting comprises microarrays and/or PCR detecting SNP polymorphisms, wherein a change in the ratio of the two alleles compared to normal tissue from the same patient indicates LOH and the presence of a precancerous lesion.

26. A kit for the detection of a precancerous lesion comprising at least one antibody to detect the phosphorylation status of a substrate of ATM and/or ATR or primers to detect LOH of a common fragile site.

27. The kit of claim 26 wherein said substrate is SMC1, Chk2, or H2AX.

28. The kit of claim 26 wherein said antibody is a phospho-specific antibody.

29. The kit of claim 26 wherein said common fragile site is FRA3B.

30. The kit of claim 26 wherein said primers are used for PCR amplification of microsatellite markers D3S1289 and/or D3S1300.
